(19) United States
(12) Patent Application Publication
(10) Pub. No.: US 2014/0113347 A1
(43) Pub. Date: Apr. 24, 2014

(54) Biopolymer Composition for Encapsulating Cells, Method for Producing a Biopolymer Composition for Encapsulating Cells, Method for Promoting Cell Cytoprotection and Use of a Biopolymer Composition for Encapsulating Cells

(71) Applicants: Ana Carolina Vale Campos Lisbôa, Barretos (BR); Gisella Grazioli, Osasco (BR); Ana Lúcia Campanha Rodrigues, Sao Paulo (BR); Leticia Labriola, Sao Paulo (BR); Mari Cleide Sogayar, Sao Paulo (BR); Thiago Rennó Dos Mares Guia, Sao Paulo (BR)

(72) Inventors: Ana Carolina Vale Campos Lisbôa, Barretos (BR); Gisella Grazioli, Osasco (BR); Ana Lúcia Campanha Rodrigues, Sao Paulo (BR); Leticia Labriola, Sao Paulo (BR); Mari Cleide Sogayar, Sao Paulo (BR); Thiago Rennó Dos Mares Guia, Sao Paulo (BR)

(73) Assignee: CELLPROTECT BIOTECNOLOGIA LTDA-ME, Sao Paulo (BR)

(21) Appl. No.: 13/994,368

(22) PCT Filed: Dec. 15, 2011
(86) PCT No.: PCT/BR2011/000479

§ 371 Date: Dec. 20, 2013

(30) Foreign Application Priority Data
Dec. 16, 2010 (BR) PII008258-1

(51) Int. Cl. C12N 11/10

(52) U.S. Cl. CPC C12N 11/10
USPC 435/178

(57) ABSTRACT

The present invention relates to a biopolymer composition for encapsulating cells, containing alginate, at least one glycosaminoglycan compound, preferably chondroitin sulfate, and at least one component of the extracellular matrix, preferably laminin, and to the process for producing the biopolymer composition. Also disclosed are a method for promoting cytoprotection using this composition, and the use of this composition for preparing a medicament useful in cell transplantation.

### FIELD OF THE INVENTION

The present invention is in the field of biotechnology and concerns a biopolymer composition for encapsulation of cells, their preparation process, a method to promote cytoprotection and the use of a biopolymer composition for the preparation of a medicament useful in transplantation cells.

### BACKGROUND OF THE INVENTION

The search for immunoprotection of transplanted cells (cytoprotection) began in 1964, when the idea of involving cells with ultrathin membranes of polymers was proposed, which has introduced the terms “artificial cells” and “bioencapsulation” in the scientific literature (Chang T M. 1964. semipermeable microcapsules. Science 146:524-525). The bioencapsulation consists of an immunoprotective barrier for cells and methodological principle aims coat cells and/or cell clusters with an artificial membrane, semipermeable, which preserves morphological and functional integrity (Calafiore R., Basta G. 1995. Microencapsulation of pancreatic islets: theoretical Principles, technologies and practice. Ricordi C, editor. Austin: RG Landes Company. p 12).

The encapsulation process must keep cells viable and protected within a membrane permeable to nutrients, ions, oxygen and other compounds necessary for the maintenance of metabolic functions, but impermeable to bacteria, lymphocytes and the macromolecules responsible for immune and inflammatory reactions, which result in rejection of the implant. There are a large number of studies in the literature reporting the use of encapsulation for immunoprotection of transplanted cells (Calafiore R. 1997. Perspectives in pancreatic and islet cell transplantation for the therapy of IDD. Diabetes Care 20 (5): 889-896; Korbutt G S, Mallett A G, the Z, Flashner, Rajotte R V. 2004. Improved survival of microencapsulated islets During in vitro culture and enhanced metabolic function Following transplantation. Diabetologia 47 (10): 1810-1818; Vos P, van Hoogmoed C G, van Zanten J, Netter S, Strubbe J H, H J Busscher. 2003. Long-term biocompatibility, chemistry, and function of microencapsulated pancreatic islets. Biomaterials 24 (2): 305-312; Campos-Lisbôa, ACV 2009. obtention of human pancreatic islets for transplantation through an Increase in cell mass and an immunoisolation with biocompatible microcapsules. 121p. PhD Thesis—Graduate Program in Biochemistry. Institute of Chemistry, University of São Paulo, São Paulo; Cornolti R, Cattaneo I, Trudu, Figliuzzi M, Remuzzi A. 2009. Islet Transplantation Effect of Glucose on Metabolic Control in Rats with diabetes. Diabetes Technology and Therapeutics, 11 (12)).

The materials used for microencapsulation have a variable composition. Two main types of materials have been studied: thermoplastic polymers and hydrogel polymers.

Studied thermoplastic polymers include poly (hydroxymethyl-acrylate-methyl methacrylate) (HEMA-MMA) acrylonitrile copolymers (AN69) and polyethylene glycol (PEG), which have advantages in relation to capsule stability after the implant. Meanwhile, the use of organic solvents, necessary for solubilizing, greatly interferes in the cell function (Vos P, Hamel A F, Tatarkiewicz K. 2002. Consideration for Successful Transplantation of encapsulated pancreatic islets. Diabetologia 45 (2): 159-173).

Among the studied hydrogels, such as alginate, chitosan and agarose, the material that better fits the standards necessary for an ideal biomaterial is the alginate, which is a polysaccharide found in both the intercellular matrix of brown algae as covering, extracellularly, some species of bacteria. Alginates are linear unbranched polymers containing residues of 1,4-β-D-manuronic (M) acid and 1,4-α-L-gulurônicos (G). These residues are interconnected in blocks of homopolymers of M (M-M-M), homopolymers G (G-G-G), heteropolymers MG, alternated (M-G-M-G) or not. Both the ratio and the distribution of the two monomers vary according to the source of the alginate and determine important physical and chemical properties for their application (Moe S T Draget K I Skja Brnk k-L, Smidsrúd O. 1995. Alginates. Stephen A M ed. New York: M Dekker. 245-286).

Among hydrogels, alginate has the greatest benefit since a) it does not interfere with the function of cells (B J de Haan, Faas M M, de Vos P. 2003. Factors influencing insulin secretion from islets encapsulated. Celi Transplantation 12 (6): 617-625), b) the preparation of the capsules occurs under physiological conditions (temperature, physiologic pH and isotonic solutions) and c) it remains stable for years in small and large animals, including humans (Soon-Shiong P Heintz R E, Merideth N, Yao Q X, Yao Z, Zheng T, Murphy M, Moloney M K, Schmehl M, Harris M, et al. 1994. Insulin independence in a type 1 diabetic patient after encapsulated islet transplantation. Lancet 343 (8903): 950-951). Moreover, this material exhibits two features that are highly desirable for the biocompatibility of a membrane: malleability and hydrophilicity. The hydrophilicity allows the surface tension between the fluid and adjacent tissue is minimal, reducing protein adsorption and cell adhesion to the biomaterial, which is undesirable for microencapsulation to restrict the diffusion of oxygen and nutrients. The flexibility of hydrogel amortizes events of mechanical irritation to the surrounding tissues (Vos P, Hamel A F Tatarkiewicz K. 2002. Considerations for successful transplantation of encapsulated pancreatic islets. Diabetologia 45 (2): 159-173). In addition, alginate changes from a soluble state to the gelled state at physiological conditions that does not affect encapsulated cells.

Alginate microcapsules are prepared by extruding a mixture of cells suspended in a solution of sodium alginate through a droplet generating device (infusion pump). Microdroplets are collected in a solution of divalent ions such as calcium or barium, becoming gel microspheres containing cells therein.

Divalent ions present in the solution gelling establish ionic bonds with the carboxyl groups present in the G blocks (homopolymer G-G-G) and in the alternating blocks MG (MG-MG or MG-GG) leading to the formation of structures called “egg boxes” (Donati I, S Holtan, Y A Morch, Borgogna M, M Dentini, Skj ak-Braek G. 2005. New hypothesis on the role of alternatxng sequences in calcium-alginate gels. Biomacromolecules 6 (2): 1031-1040) and microcapsules formation.

The technique of microencapsulation cell using different types of biopolymers have been tested (Soon-Shiong P, Heintz R E, Merideth N, Q X Yao Yao Z, Zheng T, Murphy M, Moloney M K, Schmehl M, M Harris, et al. 1994. Insulin independence in a type 1 diabetic patient after encapsulated islet transplantation. Lancet 343 (8903): 950-951; Calafiore R, Basta G, Luca G, Lemmi A, Montanucci M P, Calabrese G, Racanicchi L, Mancuso F, P. Brunetti 2006. microencapsulated pancreatic islet allografts into nonimmunosuppressed Patients with Type 1 diabetes: first two cases. Diabetes Care 29 (1): 137-138; Elliott R B, Escobar L, Tan P L Muzina M, S Zwain, Buchanan C. in 2007. Live encapsulated porcine islets from a type 1 diabetic patient 9.5 yr after xenotransplantation. Xenotransplantation 14 (2): 157-161) and applied in human clinical trials for Type 1 Diabetes Mellitus (www.reneuron.com; www.lctglobal.and with www.novocell.com) and various other diseases (Murua A, Portero A, Orive G, Hernández R M, Castro M, Pedraz J L. Cell microencapsulation technology: Towards Clinical application, 2008) anemias (Koo J, Chang T M. 1,993. Secretion of erythropoietin from microencapsulated rat kidney cells: preliminary results. The International Journal of Artificial Organs 16 (7): 557-560), stunting (Chang P L, Shen N, Westcott A J. 1993. Delivery of recombinant gene products with microencapsulated cells in vivo. Human Gene Therapy 4 (4): 433-440), hemophilia B (Hortelano G, Al-Hendy A, Ofosu F A, Chang P L. 1996. Delivery of human factor IX in mice by encapsulated recombinant myoblasts: a novel approach towards allogeneic gene therapy of hemophilia B. Blood 87 (12): 5095-5103), nephropathy (Cieslinski D A, Humes David H. 1994. Tissue engineering of a bioartificial kidney. Biotechnology and Bioengineering 43 (7): 678-681, Prakash S, Chang T M. 1996. microencapsulated genetically engineered live E. coli DH5 cells administered orally to Maintain Normal plasma urea took in uremic rats. Nature Medicine 2 (8): 883-887), hepatopathies (Wong H, Chang T M. 1986. Bioartificial liver: implanted artificial living cells microencapsulated hepatocytes Increases Survival of liver failure rats. The International Journal of Artificial Organs 9 (5): 335-336), pituitary deficiencies (Aebischer et al., 1986) and central nervous system deficiencies (Aebischer P, Panol G, Galletti P M. 1986. macroencapsulated An intraperitoneal receptacle for endocrine tissue. ASAIO Transactions/American Society for Artificial Organs Internai 32 (1): 130-133), and other diseases such as cancer (Xu W, Liu L, Charles I G. 2002. Microencapsulated Inos-Expressing Tumor Suppression cause cells in mice. Faseb J 16 (2):213-215).

Extracellular matrix components such as laminin can be used to promote mimic of extracellular matrix in microcapsules. The contact microencapsulated cells with extracellular matrix elements to guarantee the availability of a more suitable microenvironment for the viability and functionality of the graft, minimizing processes of stress and cell death if it is used as a therapy for diseases.

Studies have shown the beneficial effects of laminin and other components of the extracellular matrix in association with other biopolymers, mainly photopolymerizables, different from that one used in the present invention: it was tested the addition of laminin polyethylene glycol (PEG), followed by microencapsulation of islets pancreatic and beta cells MIN-6 from murine insulinoma, showing that the presence of this extracellular matrix element provides an improvement in the function of cells (Weber L M, Anseth K S. 2,008. Hydrogel encapsulation environments functionalized with extracellular matrix interactions Increase islet insulin secretion. Matrix Biology (8): 667-673; L M Weber, K N Hayda, Anseth K S. 2,008. Cell-Matrix B-Cell Interactions Improve Survival and Insulin Secretion in Three-Dimensional Culture. Tissue Engineering: Part A. 14 (12): 1959-1968).

The effect of the addition of pure laminin to the alginate coated with poly-L-lysine followed by microencapsulation of myoblasts is also already known (Li A A, MacDonald C N, Chang, P L. 2,003. Effect of growth factors and extracellular matrix materials on the proliferation and differentiation of microencapsulated myoblasts. J. Biomater. know. Polymer Edn 14 (6): 533-549). This work shows increased proliferation of the cells.

Laminin I also had beneficial effects when added to culture medium of human pancreatic islets in in vitro experiment of adherent monolayer culture (Labriola L, W R Montor, Krogh K Lojudice F H Genzini T, Goldberg A C Eliaschewitz F G M C Sogayar. 2007. Draw upon effects of prolactin and laminin on human pancreatic islet-cell cultures. Molecular and cellular endocrinoogy 263 (1-2): 120-133) and the work of Weber et al. (2007 and 2008) presented results in vitro of the addition of laminin in a microcapsule made from a biomaterial (PEG) different from that one used in the present invention (Weber L M, K N Hayda, Haskins K, Anseth K S. 2007. The effects of cell-matrix interactions on encapsulated beta-cell function Within hydrogels functionalized with matrix-derived adhesive peptides. Biomaterials 28, 3004-3011; Weber L M, K S Anseth. 2008. Hydrogel encapsulation environments functionalized with extracellular matrix interactions Increase islet insulin secretion. Matrix Biology 27 (8) :667-673).

Patent document WO2009/000955 describes particles of polymeric material that contains cells inside, and such particles have improved mechanical strength. This increase in resistance is achieved by functionalization of the polymeric material forming the microcapsule with the use of specific peptide which binds to cell membrane proteins. However, for effective protection against the immune response to encapsulated cells and transplanted, it is necessary to coat the microcapsules with an outer membrane that closes the pores, such as poly-L-lysine. The patent document WO2008/077402 discloses microcapsules which comprise one or more active substances embedded in a matrix in order to protect these compounds from exposure to oxygen, humidity, radiation and also against physical influences such as pressure, physical and/or chemical degradation, providing durability. Said microcapsules comprise also a complex of alginate/calcium in a ratio of about 0.1-5.0% (w/w). The microcapsules described can be used for preparing tablets and other products including an active substance.

The document WO 2007/046719 describes a composition comprising alginate, a high content of mannuronic acid and a polycation having polydispersity less than 1.5. The composition is particularly useful for the preparation of microcapsules containing living cells for transplantation type of allo- or xenogenic. Such microcapsules are superior with respect to their durability and functional and structural integrity as compared to conventional alginate capsules. The effective immunoprotection is related to the use of polycation. However, recent studies have shown that the presence of these polycations results in an activation of the immune system of the individual receiving the implant, resulting in the loss of function of transplanted microencapsulated cells. Patent document WO2003/094898 discloses biomedical materials encapsulated in alginate polymers. The alginate capsules are subjected, in a liquid vehicle, to the presence of an ethylene unsaturated monomer and an initiator so as to induce polymerization of the unsaturated monomer, and hence increase the strength of the capsule. To provide immunoprotection the microcapsules need a coat with polycations and may be further treated with poly-L-lysine to reduce their tendency to induce an immune response when implanted in an animal. To increase the strength of microcapsules, calcium ions are employed, which are lost from the medium with ease. The process for stabilizing the capsule comprises a series of steps, involving little more time in physiological solutions, as well as changes in temperature and CO₂ pressure, which may lead to a loss of viability of the cells, which are quite sensitive to these changes. Patent document WO1991/009119 describes a composition containing biological material for graft or implant, comprising alginate polymerized with barium salt, preferably barium chloride. The microcapsule may have additionally hyaluronic acid and poly-L-lysine. The microcapsule of the present invention is described as having a negative charge, which increases the release of protein and limits the invasion of immunoglobulins. Such microcapsules can be used for the encapsulation of Langerhans islets for the production of insulin. However, recent studies have shown that the use of polycations for closing pores and consequent acquisition of immunoprotection causes an undesired immune reaction around the microcapsules, jeopardizing the viability and functionality of the implant.

Although there is the state of the art reporting of large number of biopolymer compositions to confer immune protection to certain encapsulated cells, the challenge remains to keep them viable, functional and durable longevity. This challenge is the result of a number of deleterious conditions to which the cells are subjected during the encapsulation process.

Main disadvantages of the current state of the art are the high susceptibility of encapsulated cells apoptosis and cellular stress due to the conditions provided by the biomaterial coating. Although the use of polycations such as poly-L-lysine microcapsule results in a narrowing of the pore, it has been shown that is not possible to coat these polycations with alginate and that the inevitable exposure of these molecules on the surface of the microcapsules results in deleterious inflammatory cells microencapsulated (de Vos P, Faas M M, Strand B, Calafiore R. Alginate-based microcapsules for immunoisolation of pancreatic islets. Biomaterials. 2006 Nov, 27 (32):5603-17).

Furthermore, another disadvantage of the prior art, particularly with regard to the encapsulation of cell groups, such as pancreatic islets, relates to reduced insulin secretion and synthesis, mainly due to the mechanisms of apoptosis and cellular stress, as well as using a large number of cells of pancreatic islets viable for making microcapsules to a pancreatic islet transplantation in patients diagnosed with diabetes. Finally, it also detects problem as the slowness of conventional microcapsules to achieve normoglycemia after transplantation of pancreatic islet cells microencapsulated in a patient diagnosed with Diabetes.

Thus, there remains a need for biopolymer compositions which have not only good mechanical properties, but also to provide improved survival and functionality of the encapsulated cells avoiding the use of undesirable polycation, which may be prepared by processes which are less costly.

### DESCRIPTION OF FIGURES

FIG. 1 shows an experiment conducted with microcapsules prepared using a polymerization solution containing barium ions. The initial condition is given by the amount of barium ions (in ppm) present in the wash solution from the microcapsules after the production. These microcapsules were maintained in culture for 24 hours or for 7 days in a greenhouse common (static condition) and kept in rotation for 24 hours or 7 days (rotational condition) and the supernatant (culture medium) was harvested for subsequent determination of the released barium content for capsules.

FIG. 2 shows the expression of genes related to apoptosis, cellular stress, hypoxia, insulin and two samples of murine pancreatic islets microencapsulated with Alg-SC or Alg-SC-LN and maintained in culture under normoxic condition for 48 hours. Gene expression was normalized to the hprt gene whose expression is constitutive. *P<0.05, **p<0.01 and ***p<0.001. Averages of biological triplicates and experimental biological triplicate with calculation of the standard deviation in the bars for each condition. Alg-SC: Alginate+Chondroitin Sulphate, Alg-SC-LN: Alginate+Chondroitin Sulphate+Laminin.

FIG. 3 shows the blood glucose of mice with type 1 Diabetes mellitus induced by administration of streptozotocin and transplanted with 750 murine pancreatic islets microencapsulated in Alg-SC and Alg-SC-LN. As controls, it was used animals that received only naked pancreatic islets and animals that received empty capsules (sham). The graph shows the mean and standard error for each condition. The dashed line shows the limit below which the animals are considered normoglycemic. Alg-SC: Alginate+Chondroitin Sulphate, Alg-SC-LN: Alginate+Chondroitin Sulphate+Laminin.

FIG. 4 shows the oral test of glucose tolerance performed in sham control animals (transplanted with naked islets), control non-diabetic and diabetic animals transplanted with microencapsulated rat pancreatic islets with Alg-SC or Alg-SC-LN. Points represent the mean±SEM. This test was done 60 days after implantation of islets.

FIG. 5 shows the test oral glucose tolerance test performed in sham control animals (transplanted with naked islets), non-diabetic control and diabetic animals transplanted with microencapsulated rat pancreatic islets with Alg-SC or Alg-SC-LN. Points represent the mean±SEM. This test was done 150 days after implantation of islets.

FIG. 6 shows the curve of graft survival of 750 murine pancreatic islets microencapsulated with Alg-SC or Alg-SC-LN transplanted into mice with Diabetes mellitus type 1 induced by administration of streptozotocin. Alg-SC: Alginate+Chondroitin Sulphate, SC-LN-Alg: Alginate+Chondroitin Sulphate+Laminin. The results show that even after 200 days biomaterial graft functionality of SC-Alg-LN remains in about 60% of the transplanted animals and it is significantly higher than the functionality of the graft microencapsulated with Alg-SC. Mantel-Cox test, * p<0.05.

FIG. 7 shows the evaluation of the biocompatibility of the biopolymers Alg-SC and Alg-SC-LN in view of microcapsules incubation with macrophage cell line RAW 264-7 and analysis of RNA expression level of IL-l-beta (FIG. 7A) and TNF-alpha (FIG. 7B) expressed by macrophages after 3 hours, 9 hours and 24 hours of incubation. It was used the housekeeping gene HPRT for normalization of data. It was used as negative control macrophages incubated without microcapsules. As a positive control, macrophages with LPS (bacterial lipopolysaccharide) and non-purified biopolymer for clinical use (Alg-Sigma). Results demonstrate the absence of macrophage activation (cytokine expression) against contact with biomaterials Alg-SC and Alg-SC-LN, showing that they are biocompatible, non-immunogenic. Biological triplicates with experimental duplicates. Test One-Way ANOVA with Tukey post test. ** P<0.01. Alg-SC: Alginate+Chondroitin Sulphate, SC-LN-Alg: Alginate+Chondroitin Sulphate+Laminin.

FIG. 8 shows the relative protein expression of Bax proteins (FIG. 8A), Bcl-xL (FIG. 8B) and XIAP (FIG. 8C) in microencapsulated islets with Alg-SC or Alg-SC-LN by Western blot, normalized according to the expression of GAPDH protein. * P<0.05 and ** p<0.01. Alg-SC: Alginate+Chondroitin Sulphate, SC-LN-Alg: Alginate+Chondroitin Sulphate+Laminin.

### DESCRIPTION OF THE INVENTION

The combined deficient factors found in the prior art for the encapsulation of cells led to the development of a new biopolymer composition for the encapsulation of cells which not only presents improved mechanical strength but also improves the survival and function of the encapsulated cells, or presents higher biocompatibility and stability compared to other microcapsules presented in the literature. These attributes are achieved with the use of a specific combination of components, in specific amounts that eliminate the need of the use of polycations and avoid undesirable complicated methods of production.

The present invention also relates to a method of producing said composition by specifically adjusted parameters that guarantee the efficiency of the composition in a shorter production time. Still objects of the present invention are a method for promoting the cytoprotection and a use of the developed biopolymer composition for the manufacture of a medicament useful for cell transplantation.

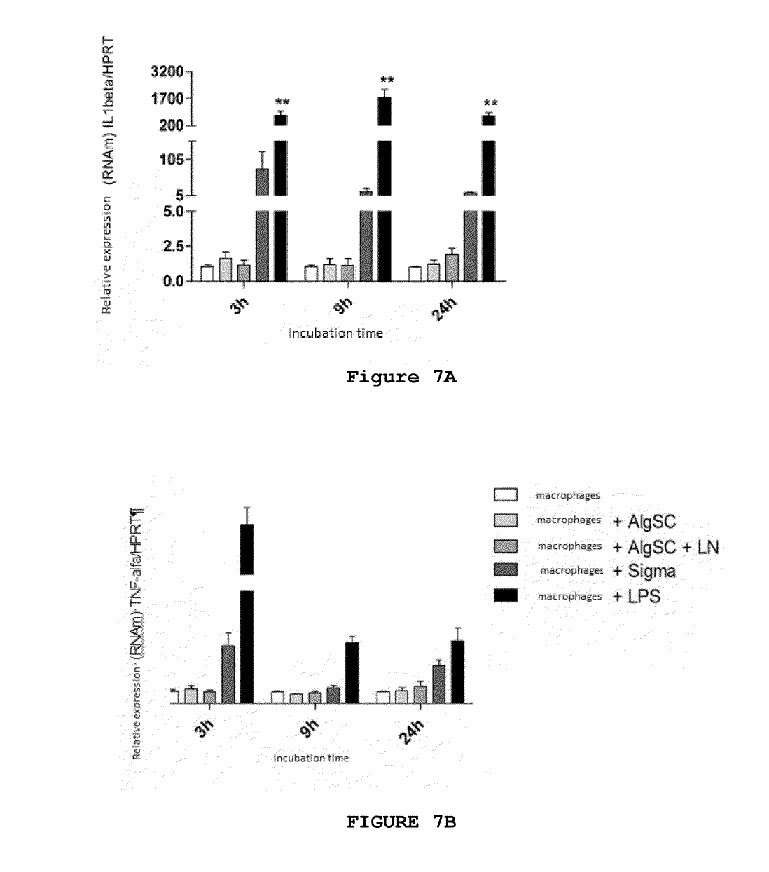

It was found in this new biopolymer composition superior properties and benefits that ensure the biocompatibility (FIG. 7), the function and viability of microencapsulated cells, which is essential not only for therapeutic or prophylactic activities are achieved, but also to ensure that maximum longevity and viability of the transplanted cells.

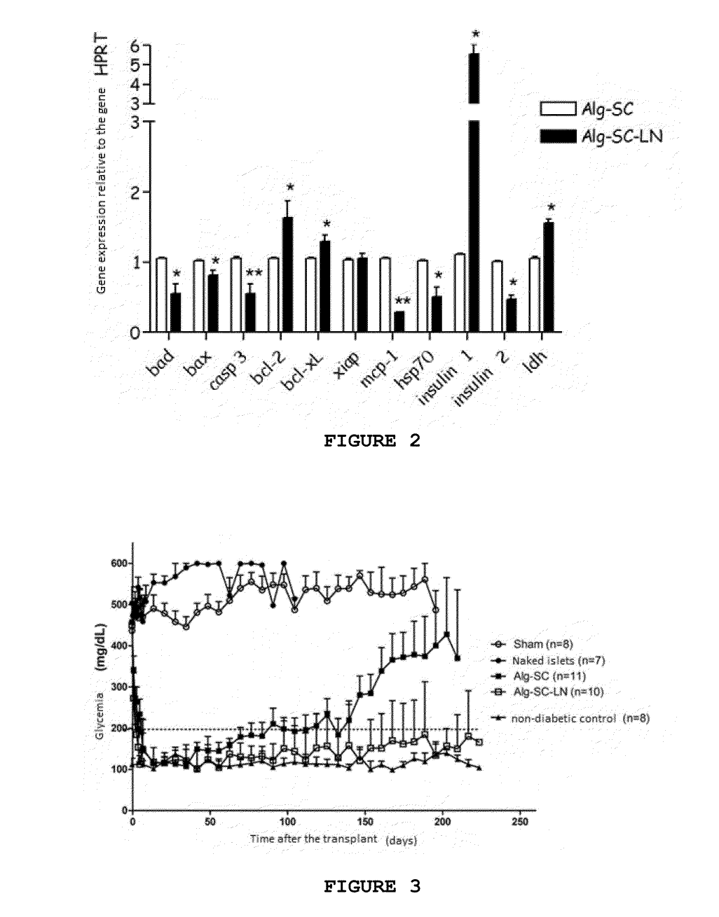

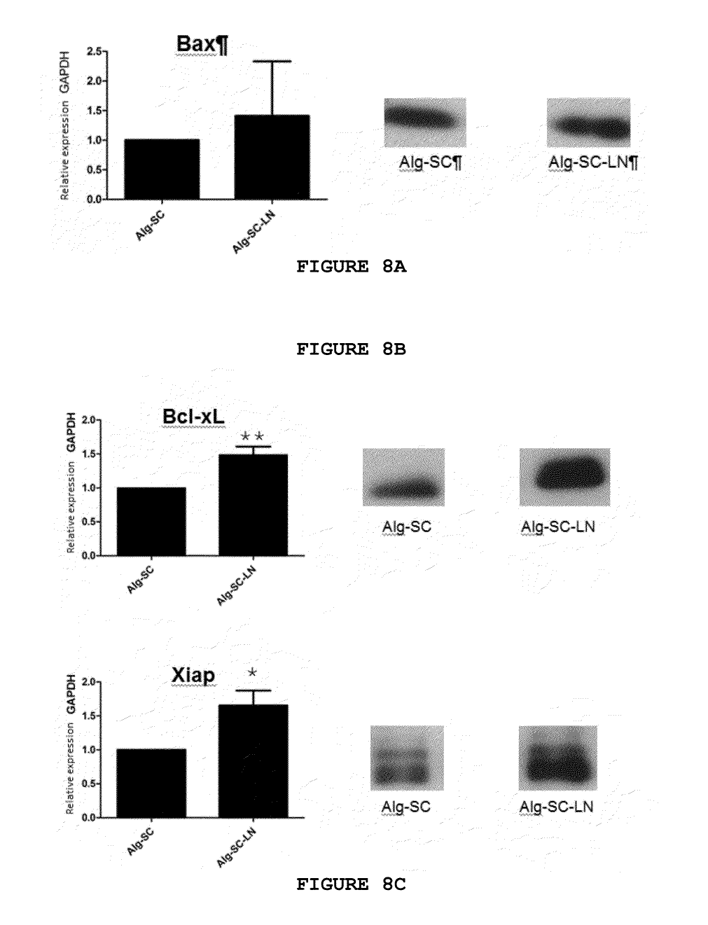

The researchers of the present invention found that the apoptosis and cellular stress can be reduced by adding elements of the extracellular matrix such as laminin in biopolymers systems based on alginate and chondroitin sulfate, in order to prevent cell death and also promote proliferation and cell viability (FIGS. 2 and 8).

In addition, the same researchers found that addition of chondroitin sulfate and laminin to the composition of the biopolymer alginate ensures narrowing of the pores and protection of microencapsulated cells of molecules and cells of the immune system, solving the problem that the micro-encapsulation with alginate gelled with calcium creates pores to produce microcapsules with larger dimensions. This situation increases the resistance of the microcapsules and improves survival and functionality of the encapsulated cells through cytoprotective molecular signals (FIGS. 2 and 8).

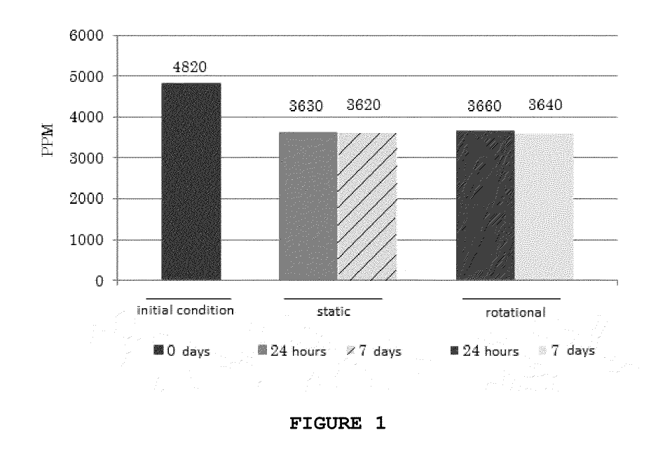

The combination of alginate, glycosaminoglycans components, such as chondroitin sulfate, and extracellular matrix components such as laminin, for the manufacture of microcapsules which are gelled by solution of divalent cations, such as barium ions, was introduced as a great improvement over the current state of the art by adding biological properties advantageous to the microcapsules. Even barium compound is toxic to the body once it establishes ionic bonds within the microcapsule mesh it becomes unavailable to the surroundings and thus no significant toxicity to the individual receiving the implant and the encapsulated cells themselves (FIG. 1).

The current state of the art does not disclose compositions for the encapsulation of biopolymer-based cells in alginate and glycosaminoglycan components of the extracellular matrix components. Furthermore, there are known methods for promoting cytoprotection using such compositions, as well as the use of these particular compositions for the preparation of a medicament useful in cell transplantation.

One of the objects of this invention relates to a biopolymer composition for the encapsulation of cells based on alginate, glycosaminoglycans components, such as chondroitin sulfate, and extracellular matrix components.

The components of the extracellular matrix may be one or more of elastin, entactin-1, fibrillin, fibronectin, fibrin, fibrinogen, fibroglycan, fibromodulin, fibulin, glypican, vitronectin, laminin, nidogen, matrilin, perlecan, heparin, heparan sulfate, heparan sulfate proteoglycans, decorin, filaggrin, keratin, syndecan, agrin, integrin, aggrecan, biglycan, hyaluronan, the hyaluronan binding proteins, serglycin, tenascin, nidogen, chondronectin, thrombospondin, versican, hb-gam, dermatan sulfate, keratan sulfate, collagens (including types IV and XVIII), fibrillar collagens (including types I, II, III, V and XI), FACIT collagens (types IX, XII, XIV), other collagens (types VI, VII, XIII), short-chain collagens (types VIII and X), chondroitin sulfate (including the types a, c, d, e), lumican and domains, chains, fragments, mutants or analogs thereof. In a particular way the extracellular matrix component is laminin.

In the composition according to the present invention the ratio of alginate:chondroitin sulfate is about 4:1 and laminin is present in a final concentration of about 10 μg·mL⁻¹.

Another object of this invention relates to a method for promoting the protection of encapsulated cells (cytoprotection) by using a biopolymer composition based on alginate, glycosaminoglycans components, such as chondroitin, and extracellular matrix components such as laminin, according with the present invention.

It is also another object of this invention to the use of a composition based on alginate biopolymer, components glycosaminoglycans such as chondroitin and extracellular matrix components such as laminin, according to the present invention for the preparation of a medicament useful in cell transplantation.

There are several advantages of the present invention over the prior art, the same being scored as follows:

1) developed biopolymer composition induces changes in the expression of important genes related to apoptosis. The effector caspase-3 gene, which is activated late in the apoptotic cascade, has its reduced expression in cells microencapsulated biopolymer with the composition of this invention. In addition, the anti-apoptotic bcl-2 gene, whose product is important for protecting cells against apoptosis mechanisms has its increased expression in cells microencapsulated with said composition (FIG. 2). The expression of anti-apoptotic proteins Bcl-xL and XIAP are also elevated in microencapsulated islets with Alg-SC-LN microencapsulated islets compared with Alg-SC (FIG. 8).

2) The claimed composition biopolymer increases the ratio of gene expression of bcl-2 and bax (bcl-2/bax), which shows that microencapsulation with such a composition protects cells against apoptosis. Increased ratios of the expression of bcl-2/bax and bcl-xL/bax at both the gene and protein level, showed a decrease in the susceptibility of the cell to apoptosis (Brown et al., 2007) (FIG. 2).

3) The developed biopolymer composition decreases expression of the genes MCP-1 and hsp70, both related to cellular stress (FIG. 2).

4) The developed composition biopolymer in pancreatic islet cells microencapsulation models increases the expression of the rat insulin 1 gene, which can lead to an increase in the percentage of β cell precursors which differentiate into mature β cells. This composition also restores cell-cell and cell-matrix contact that is essential for maintenance of cell viability and function (FIG. 2).

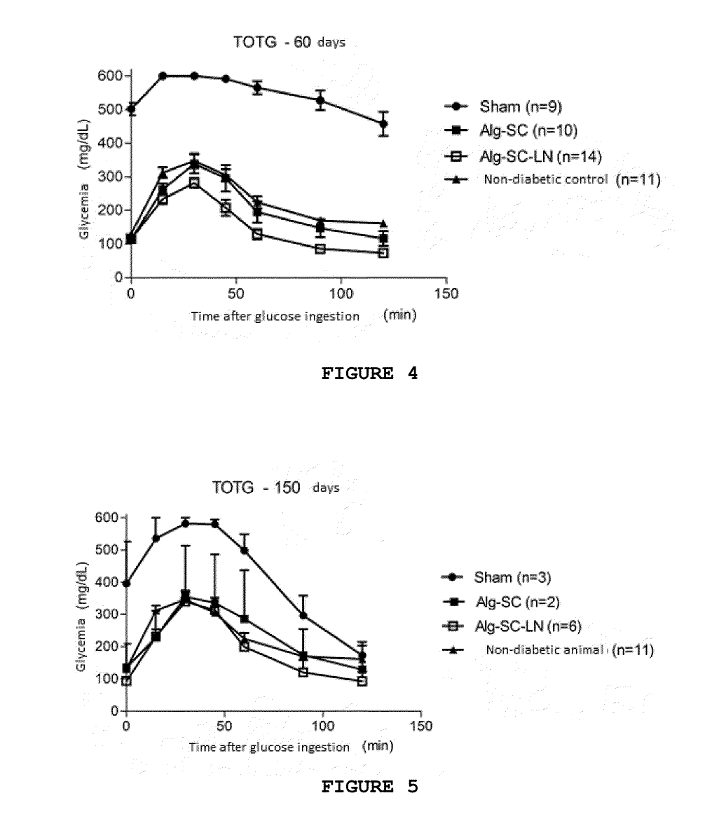

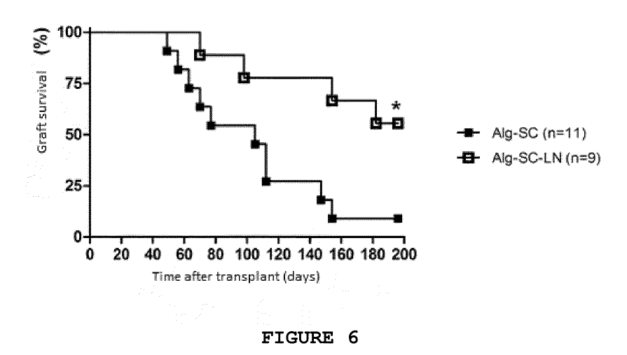

5) The developed method for cell encapsulation uses a reduced number of pancreatic islet cells required for cell transplantation. For the current state of the art, the mean number of rat islets required to reverse the mice diabetic state is about 1,450. By using the biopolymer composition of the present invention, it was possible to maintain normoglycemia in diabetic mice over 200 days via an implant of only 750 microencapsulated islets, which is 48% less than the amount islets reported in the art (FIG. 3). The longevity of the graft was significantly higher in mice that received microencapsulated islets with Alg-SC-LN (FIG. 6). The quality of the islets was assessed by transplantation throughout the test oral glucose tolerance test (OGTT) (FIGS. 4 and 5). This difference is significant when considering that this reduction can lead to a pancreas saving for islet transplantation in humans, since the average surgeries require islets (encapsulated or not) extracted from two or more human pancreas in order to the diabetic patient receiver has normalized blood glucose.

6) In pancreatic islet cell microencapsulation model, the developed biopolymer composition of the present invention gets normoglycemia after transplantation in a shorter period of time compared to the current level of technology. The presence of extracellular matrix components such as laminin in the composition of this invention reduces by up to 4 days to reach normoglycemia after transplantation. In the clinic, this can mean fewer days of hospitalization due to a faster recovery of the patient (FIG. 3).

7) The biocompatibility of biomaterials Alg-SC and Alg-SC-LN was demonstrated by testing in which these biomaterials-containing capsules were placed in culture with macrophages. The expression of pro-inflammatory cytokines IL-l-beta and TNF-alpha was not stimulated in the macrophages cultured with these biomaterials in contrast with the increased expression of these cytokines in macrophages cultured with alginate grom Sigma manufacturer or in the presence of LPS (FIG. 7.)

Alternative forms or modifications within the scope of the present invention will become readily detectable by person skilled in the art from reading the following specifications and references.

The method of producing the biopolymer composition consists in the mixture of ideal proportions of alginate with at least one component glycosaminoglycan, preferably chondroitin sulphate, together with at least one extracellular matrix component, preferably laminin.

This mixing is done at the time of microencapsulation, as well as mixing the biomaterial with cells. This final mixture should be performed as quickly as possible because of the time consuming and direct contact with biomaterial ungelated cells can cause harmful effects to the viability and functionality of these. This procedure should be performed only when the entire apparatus of microencapsulation is prepared for making the microcapsules. The cells should be sedimented by centrifugation, and homogenized thoroughly in the biomaterial, and adding this mixture to a syringe, which is connected to the device that produces the microcapsules.

The cell encapsulation may be directed to stem cells, muscle cells, pancreatic cells, chondrocytes, liver cells, cells of the central nervous system, renal cortex cells, vascular endothelial cells, skin cells, parathyroid and thyroid cells, adrenal cells, cells thymic cells, ovarian germline cells, embryos or cells which include recombinant genetic material.

For making the microcapsules, it uses a syringe pump to expel the mixture of the biopolymer with the cells. By applying an air flow around the coaxial needle, it is possible to detach the droplet at the desired time, and therefore control the size of the microcapsules. The distance between the needle tip and the gelling solution is adjusted to microcapsules delicately reach the bottom of the container where they are deposited, thus avoiding mechanical shock which can cause deformations. The height between the needle tip and exits the biomaterial containing the cells and the gelling solution may be between 5 and 10 cm. The flow of biomaterial containing the cells can be expelled through the needle at a flow ranging between 15 and 30 mL·h⁻¹. The coaxial air flow can vary between 2.0 and 2.5 L·min⁻¹, which can generate microcapsules still considered optimum between 500 and 1000 μm.

The diameter of the microcapsules is dependent on the ion used for the gel formation, on the gel solution concentration and the flow of air. After detachment of the needle, the microcapsules fall into a solution of polymerization (gel formation) comprising divalent ions, such as BaCl₂ or CaCl₂, preferably BaCl₂, and it is buffered with 4-(2-hydroxyethyl)-1-piperazineethanesulfonic acid—HEPES at pH 7.4.

It is important to note that gel formation is conducted under physiological conditions, causing no harm to the cells. When it comes into contact with the gelling solution, the biomaterial passes from the soluble state to gel state. Laminin and chondroitin sulfate, which are contained in the biomaterial, remain anchored in the alginate-ion networks, helping to close the formed mesh and forming pores of suitable size.

At the end of the process, the microcapsules remain in the solution for 5 minutes. After this incubation step, the microcapsules are rapidly filtered. The excess ion used for the gelation of the biomaterial is removed by successive washing of the microcapsules in 0.15 M NaCl.

The microcapsule formed around the cells is permeable to insulin, glucose, nutrients and oxygen and impermeable to molecules and cells of the immune system, preventing direct contact between the transplanted graft and the patient's immune system in case of cell transplantation or cell therapy.

Other application techniques can also be attributed to the invention as the large-scale production of molecules derived from cells, reproductive technology and cell culture-dependent contact with other cells and/or proteins, and others, such as the food industry (microencapsulation of yeast for brewing beer and microencapsulation of seeds) and in the pharmaceutical industry (microencapsulation of biopharmaceuticals or chemotherapy).

The following examples are provided in order to illustrate the main aspects of the present invention. It should be noted that for those who know the state of the art, the descriptions below, used by the inventors of this invention may be regarded as one of the various ways in which the invention can be achieved. It is understood that changes invention can be used in effect, obtaining also results equal or similar to those described in the scope of this invention. The invention is further explained by the following examples.

### EXAMPLES

### Example 1

### Preparation of the Biomaterial of the Biomaterial and Mixed with the Cells Whether to Microencapsulate

The biopolymer is diluted in NaCl 0.15 mol L⁻¹ to a final concentration of 1.2% alginate. The alginate biopolymer is formed by alginate:chondroitin sulfate in a ratio of 4:1 and laminin-1 is added in this mixture to a final concentration of 10 μg·mL⁻¹. The cell suspension should be carefully and fast homogenized in the solution of biopolymer-NaCl. The gelling solution is 0.02 mol·L⁻¹ Barium Chloride plus 20 mmol·L⁻¹ HEPES (Sigma), pH 7.2.

### Example 2

### Preparation of Microcapsules and Parameters Used to Obtain a Microcapsule with Good Size, Shape and Stability

10,000 islets are used per mL alginate, laminin, chondroitin sulfate or 1.5×10⁶ cells·mL⁻¹ biopolymer. The capsules were obtained by extruding the solution containing the biomaterial islets (or cells) by a microneedle at a flow rate of 19.9 mL·h⁻¹ controlled by a syringe pump (SP 500 JMS do Brasil, Campinas, Brazil). By applying 2.2 L·min⁻¹ air flow (air medicinal, Air Products Brasil Ltda.) around the needle. After detachment of the needle drop, the microcapsules falls into a polymerization solution (gelling) comprising BaCl₂. With the above determined flow it is obtained microcapsules with a diameter of about 700-800 μm.

The distance between the needle tip and the gelling solution was adjusted to 7.5 cm. At the end of the process, the microcapsules remain in the solution for 5 minutes. After this incubation step, the microcapsules are rapidly filtered and washed with 0.15 mol·L⁻¹ NaCl.

1. A biopolymer composition for encapsulating cells comprising alginate, at least one glycosaminoglycan component and at least one extracellular matrix component at a final concentration of about 10 μg·mL⁻¹.

2. The biopolymer composition according to claim 1 wherein the glycosaminoglycan component is chondroitin sulfate.

3. The biopolymer composition according to claim 1 wherein extracellular matrix component is one or more of elastin, entactin-1, fibrillin, fibronectin, fibrin, fibrinogen, fibroglycan, fibromodulin, fibulin, glypican, vitronectin, laminin, nidogen, matrilin, perlecan, heparin, heparan sulfate, heparan sulfate proteoglycans, decorin, filaggrin, keratin, syndecan, agrin, integrin, aggrecan, biglycan, hyaluronan, hyaluronan binding proteins, serglycin, tenascin, nidogen, chondronectin, thrombospondin, versican, hb-gam, dermatan sulfate, keratan sulfate, collagens (including types IV and XVIII), fibrillar collagens (including types I, II, III, V and XI), FACIT collagens (types IX, XII, XIV), other collagens (types VI, VII, XIII), short-chain collagens (types VIII and X), or lumican, and domains, chains, fragments, mutants or analogs thereof.

4. The biopolymer composition according to claim 1 wherein the ratio of alginate:glycosaminoglycan component is about 4:1

5. The biopolymer composition according to claim 3 wherein laminin is present in a final concentration of about 10 μg·mL⁻¹.

6. The biopolymer composition according to claim 1 wherein the cells are selected from one or more of stem cells, muscle cells, pancreatic cells, chondrocytes, liver cells, central nervous system cells, kidney cortex cells, endothelial vascular cells, skin cells, thyroid. and parathyroid cells, adrenal cells, thymic cells, ovarian cells, germline cells, embryos or cells that include recombinant genetic material.

7. A composition according to claim 6 comprising about 1.5×10⁶ cells·mL⁻¹ of the biopolymer composition.

8. A method for production of a biopolymer composition for cell encapsulation comprising: (a) mixing alginate at a concentration of about 1.2% with a glycosaminoglycan component at a ratio of about 4:1, (b) adding one extracellular matrix component to a final concentration of about 10 μg·ml⁻¹ and (c) promote polymerization using divalent cations.

9. The method according to claim 8 wherein the divalent cation is selected from calcium or barium ions.

10. The method according to claim 8 wherein it is added about 1.5×10⁶ cells·mL⁻¹ of composition.

11. The method according to claim 10 wherein cells are selected from one or more of stem cells, muscle cells, pancreatic cells, chondrocytes, liver cells, central nervous system cells, renal cortex cells, vascular endothelial cells, skin cells, thyroid and parathyroid cells, adrenal cells, thymic cells, ovarian cells, germline cells, embryos or cells that include recombinant genetic material.

12. A method for promoting cytoprotection comprising the encapsulation of cells with the biopolymer composition according to claim 1.

13. The method according to claim 12. wherein cells are selected from one or more of stem cells, muscle cells, pancreatic cells, chondrocytes, liver cells, central nervous system cells, renal cortex cells, vascular endothelial cells, skin cells, thyroid and parathyroid cells, adrenal cells, thymic cells, ovarian cells, germline cells, embryos or cells that include recombinant genetic material.

14. (canceled)
